(19) United States
(12) Plant Patent Application Publication
(10) Pub. No.: US 2020/0337194 P1
(43) Pub. Date: Oct. 22, 2020

(54) Strawberry plant named 'DRISSTRAWSEVENTYONE'

(50) ;

(71) Applicant: Driscoll's, Inc., Watsonville, CA (US)

(72) Inventors: Philip J. Stewart, Watsonville, CA (US); Joanne F. Coss, Watsonville, CA (US); Kevin Coons, Watsonville, CA (US); Amy Marie Edmondson, Watsonville, CA (US); Iana Kostina, Watsonville, CA (US)

(73) Assignee: Driscoll's, Inc., Watsonville, CA (US)

(21) Appl. No.: 16/501,449

(22) Filed: Apr. 17, 2019

(51) Int. Cl. A01H 6/74

(52) U.S. Cl. PLT/209

(57) ABSTRACT

A new and distinct variety of strawberry plant named ‘DrisStrawSeventyOne’, selected for its open habit, plant health, fruit appearance, and fruit flavor, is disclosed.

Latin name: Botanical classification: Fragaria x ananassa.

Varietal denomination: The varietal denomination of the claimed variety of strawberry plant is ‘DrisStrawSeventyOne’.

### BACKGROUND OF THE INVENTION

Cultivated strawberry is a hybrid species of the genus Fragaria that is grown worldwide for its fruit. Modern strawberry was first bred in Brittany, France, in the 18^{th} century by crossing Fragaria virginiana with Fragaria chiloensis. Strawberry fruit is an aggregate accessory fruit, with the fleshy part of the fruit being derived from the receptacle that holds the ovaries.

Strawberry varieties vary widely in color, size, shape, flavor, season of ripening, degree of fertility, and susceptibility to disease. Certain varieties vary in foliage, and some vary in the relative development of their reproductive organs. Typically, strawberry flowers appear hermaphroditic in structure, but function as either male or female. Generally, commercial production of strawberry plants involves propagation from runners and distribution as either plugs or bare root plants. Cultivation is either perennial or annual plasticulture. During the off season, strawberries can also be produced in greenhouses.

Strawberry fruit is widely appreciated for its characteristic bright red color, aroma, juicy texture, and sweetness. Strawberry fruit is a popular fruit that is generally consumed either fresh or in prepared foods, such as preserves and baked goods.

Strawberry is an important and valuable fruit crop. Accordingly, there is a need for new varieties of strawberry plants. In particular, there is a need for improved varieties of strawberry plant that are stable, high yielding, and agronomically sound.

### SUMMARY OF THE INVENTION

In order to meet these needs, the present invention is directed to an improved variety of strawberry plant. In particular, the invention relates to a new and distinct variety of strawberry plant (Fragaria x ananassa), which has been denominated as ‘DrisStrawSeventyOne’.

Strawberry plant variety ‘DrisStrawSeventyOne’ originated from a cross between the proprietary female parent ‘35T 42’ (unpatented) and the proprietary male parent ‘50T403’ (unpatented). Progeny plants from this cross, including ‘DrisStrawSeventyOne’, were asexually propagated via stolons in McArthur, Shasta County, Calif. in April of 2012. Strawberry plant variety ‘DrisStrawSeventyOne’ was later specifically identified and selected in Monterey County, Calif. in May of 2013.

‘DrisStrawSeventyOne’ was subsequently asexually propagated via stolons, and underwent further testing at a farm in Monterey County, Calif. for four years (2013 to 2017). The present variety has been found to be stable and reproduce true to type through successive asexual propagations via stolons.

‘DrisStrawSeventyOne’ exhibits the following distinguishing characteristics when grown under normal horticultural practices in Monterey County, Calif.:
1. Inflorescence beneath foliage;
2. Obtuse shape of base of terminal leaflet; and
3. Partially remontant type of bearing.

‘DrisStrawSeventyOne’ was selected for its growth habit, plant health, fruit appearance, and fruit flavor.

### DESCRIPTION OF THE DRAWINGS

This new strawberry plant is illustrated by the accompanying photographs which show fruit of the plant, as well as the flowers, leaves and plants. The colors shown are as true as can be reasonably obtained by conventional photographic procedures. The photographs are of plants that are ten months old.

FIG. 1 illustrates whole fruit of variety ‘DrisStrawSeventyOne’.

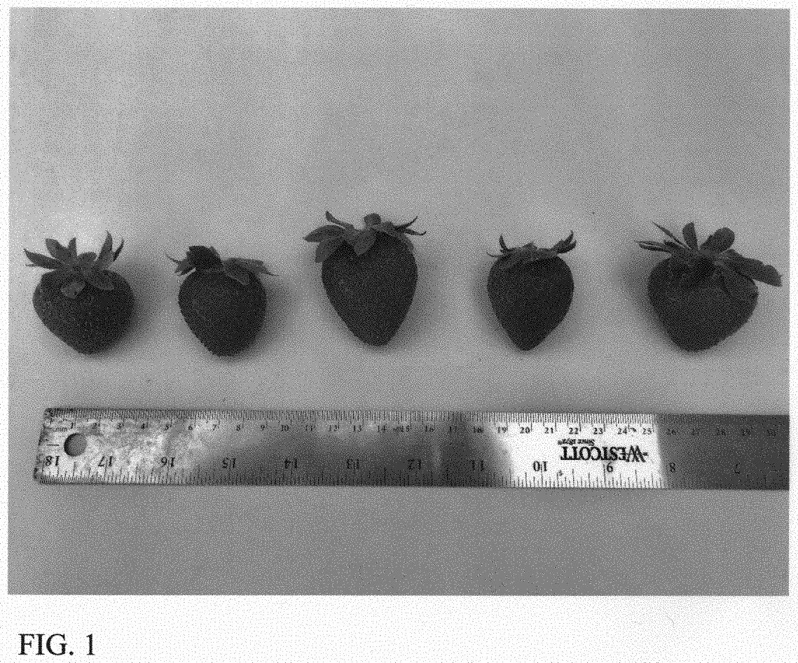

FIG. 2 illustrates longitudinal sections of fruit of variety ‘DrisStrawSeventyOne’.

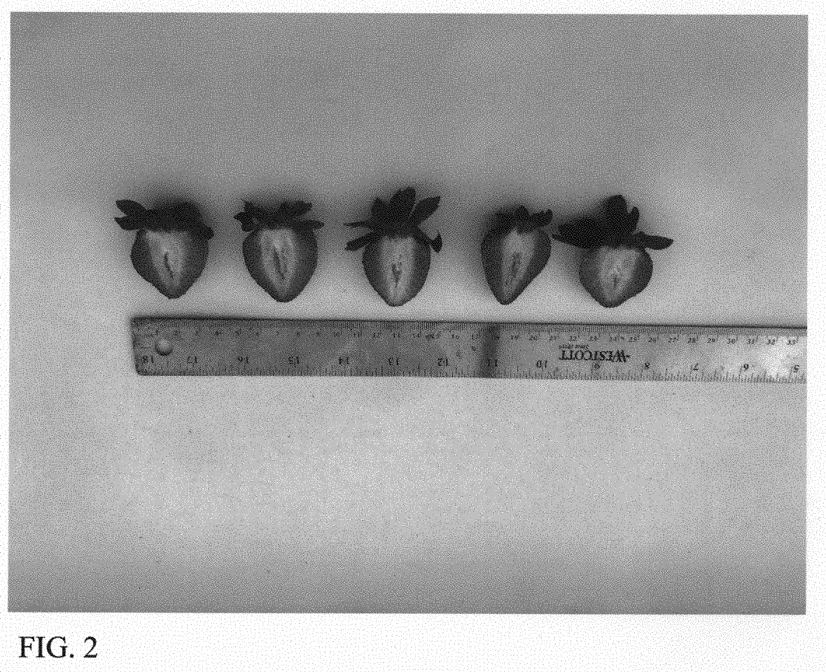

FIG. 3 shows the upper and lower surfaces of flowers of variety ‘DrisStrawSeventyOne’.

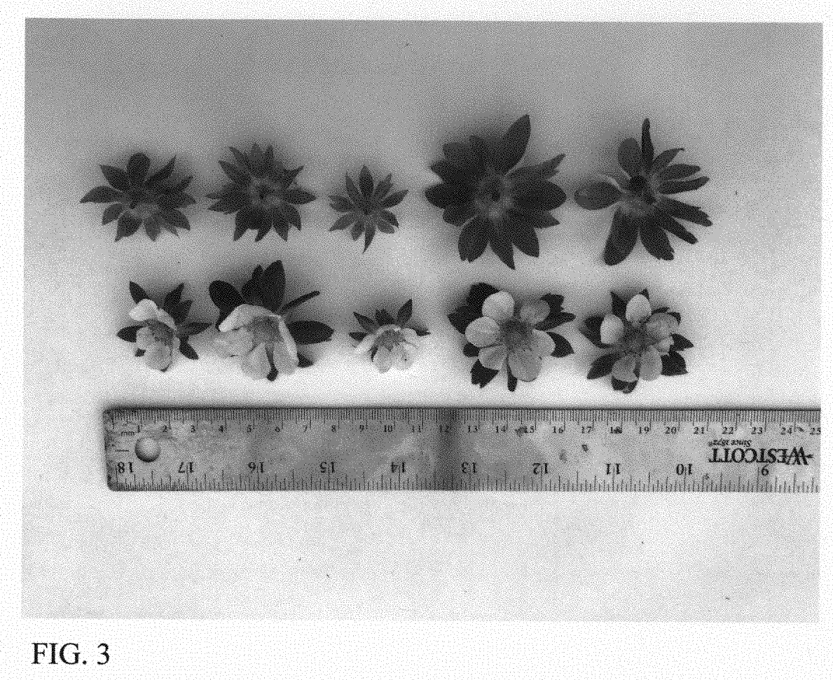

FIG. 4 show leaves of variety ‘DrisStrawSeventyOne’.

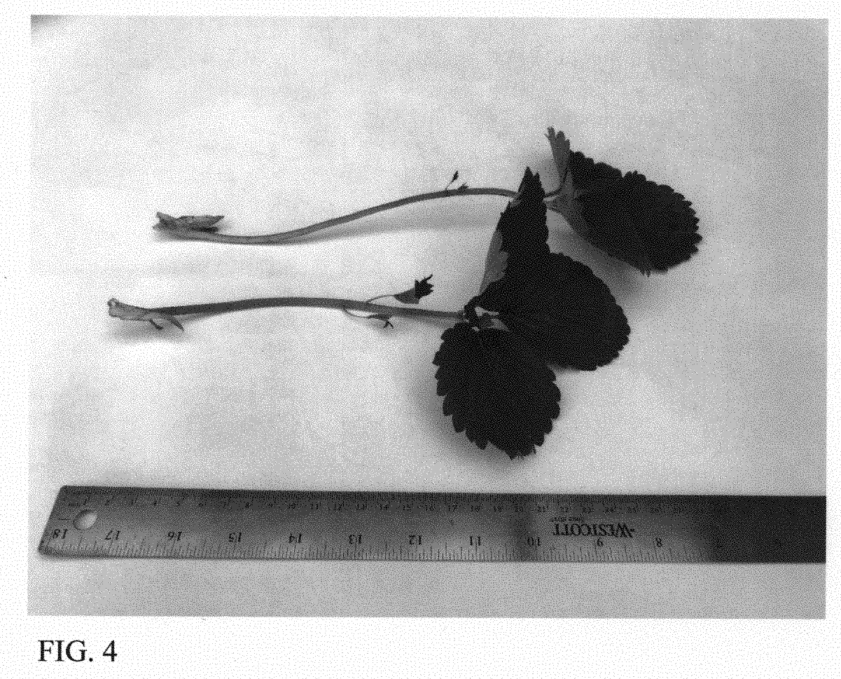

FIG. 5 shows plants of variety ‘DrisStrawSeventyOne’.

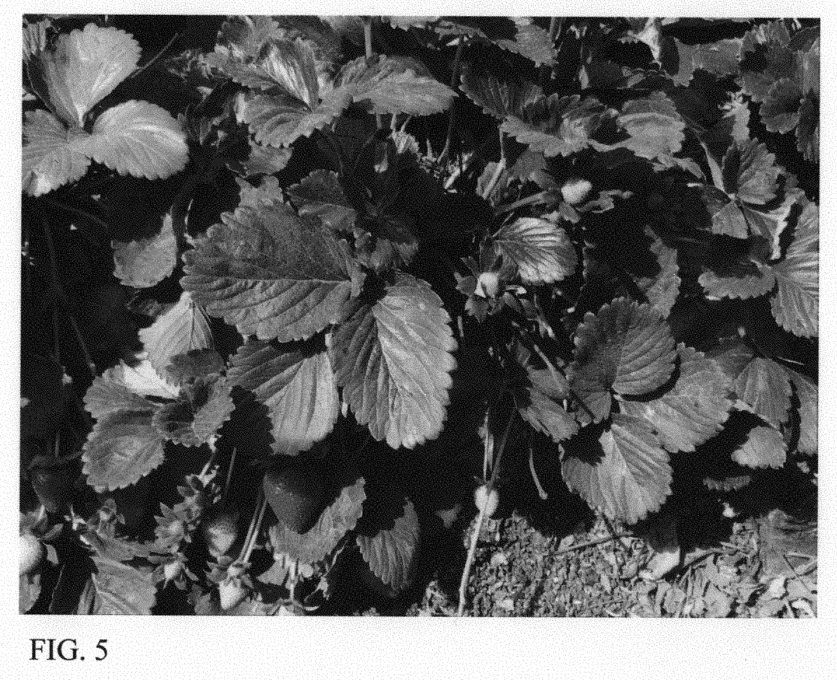

### DETAILED BOTANICAL DESCRIPTION OF THE PLANT

The following detailed descriptions set forth the distinctive characteristics of ‘DrisStrawSeventyOne’. The data which define these characteristics is based on observations taken in Monterey County, Calif. from 2013 to 2017. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawSeventyOne’ has not been observed under all possible environmental conditions. The botanical description of ‘DrisStrawSeventyOne’ was taken from plants that were ten months old. The indicated values represent averages calculated from measurements of several plants. Color references are primarily to The R.H.S. Colour Chart of The Royal Horticultural Society of London (R.H.S.) (2015 edition). Descriptive terminology follows the Plant Identification Terminology, An Illustrated Glossary, 2^{nd} edition by James G. Harris and Melinda Woolf Harris, unless where otherwise defined.
Classification:
   Species.—Fragaria x ananassa.
   Common name.—Strawberry.
   Denomination.—‘DrisStrawSeventyOne’.
Parentage:
   Female parent.—The proprietary variety ‘35T 42’ (unpatented).
   Male parent.—The proprietary variety ‘50T403’ (unpatented).
Plant:
   Height.—32.15 cm.
   Diameter.—44.1 cm.
   Number of crowns per plant.—8.5.
   Growth habit.—Semi-upright.
   Density of foliage.—Medium.
   Vigor.—Medium.
Stolon:
   Average number of daughter plants per square foot.—20.
   Diameter at bract.—3.73 mm.
   Density of pubescence.—Medium.
   Stolon color.—RHS 144B (Strong yellow-green).
   Anthocyanin coloration.—Absent or very weak.
   Anthocyanin color.—RHS 140C (Brilliant yellowish green).
Leaf:
   Number of leaflets.—Three only.
   Color of upper surface.—RHS 139A (Dark Yellowish Green).
   Color of lower surface.—RHS 138B (Moderate yellow-green).
   Blistering.—Medium.
   Glossiness.—Medium.
   Variegation.—Absent.
   Terminal Leaflets.—Length: 8.49 cm. Width: 7.40 cm. Length/width ratio: 1.15 . Number of teeth/terminal leaflet: 24.5 . Shape of base: Obtuse. Margin: Serrate to crenate. Shape in cross section: Concave.
   Petiole.—Length: 25.1 cm. Diameter: 4.07 mm. Attitude of hairs: Slightly outwards. Bract frequency (number present on each petiole): 1.25 . Petiole color: RHS 144C (Strong yellow-green).
   Petiolule.—Length: 25.55 mm. Diameter: 2.32 mm.
   Stipule.—Length: 3.86 cm. Width: 16.63 mm. Anthocyanin coloration: Weak. Anthocyanin color: RHS N144D (Strong yellow green). Stipule color: RHS 139A (Darkish yellow-green).
Inflorescence:
   Position in relation to foliage.—Beneath.
   Pedicel.—Attitude of hairs: Slightly outwards. Pedicel color: RHS 143D (Moderate yellow-green).
   Flower.—Flower diameter (petal tip to petal tip on non-flattened flower): 30.86 mm. Arrangement of petals: Free. Stamen: Present. Stamen length: 1.0 mm. Stamen width: 0.5 mm. Stamen shape: Basifixed. Stamen color: RHS 150A (Brilliant yellow-green). Number of stamen per flower: 28.2 . Anther color: RHS 164B (Brownish orange). Typical and observed number of flowers per plant: 9.5 .
   Petal.—Length: 12.41 mm. Width: 10.61 mm. Length/width ratio: 1.17 . Typical and observed petal number: 5.6 . Color of upper side: RHS NN155D (White). Color of lower side: RHS 155A (Pale yellow-green).
   Calyx.—Diameter (sepal tip to sepal tip, measured on back of flower): 42.45 mm. Calyx color: RHS 141D (Strong yellow-green).
   Sepal.—Length (sepal tip to point of attachment to receptacle): 16.5 mm. Width: 8.74 mm. Typical and observed sepal number: 11.7.
Fruit:
   Length.—41.32 mm.
   Width.—36.45 mm.
   Length/width ratio.—1.13.
   Fruit hollow length.—18.42 mm.
   Fruit hollow width.—5.93 mm.
   Fruit hollow length/width ratio.—3.11.
   Shape.—Conical.
   Differences in shape between primary and secondary fruit.—Slight.
   Color.—RHS N45A (Moderate Red).
   Number of fruit per truss.—3.0.
   Evenness of color.—Even or very slightly uneven.
   Glossiness.—Strong.
   Width of the band without achenes.—Narrow.
   Firmness.—Medium.
   Achenes.—Position of achenes: Level with surface. Achene color: RHS 150A (Brilliant yellow-green). Number of achenes per fruit: 306. Achene weight: 0.5 mg.
   Peduncle.—Peduncle length: 7.0 cm. Peduncle diameter: 2.0 mm.
   Position of calyx attachment.—Level with fruit.
   Attitude of sepals.—Outwards.
   Color of flesh (excluding core).—RHS 40A (Vivid reddish orange).
   Color of core.—RHS 39B (Deep yellowish pink).
   Titratable acidity (as citric acid content).—0.74%.
   Sugar content (as soluble solids in Brix).—8.1°.
Production:
   Flowering interval.—April to October.
   Harvest interval.—April to October.
   Type of bearing.—Partially remontant.
   Productivity.—: 34,279 kg to 51,363 kg of fruit per acre per season from ten-month-old plants when grown in Monterey County, Calif.
Resistance to diseases, pests, and abiotic stress:
   Heat.—Moderately susceptible.
   Botrytis fruit rot (Botrytis cinerea).—Moderately susceptible.
   Powdery mildew (Podosphaera macularis).—Moderately resistant.
   Anthracnose crown rot (Colletotrichum acutatum).—Susceptible.

### COMPARISON WITH PARENTAL AND COMMERCIAL VARIETIES

‘DrisStrawSeventyOne’ differs from the proprietary female parent ‘35T 42’ (unpatented) in that ‘DrisStrawSeventyOne’ has larger fruit compared to ‘35T 42’. Moreover, plants of ‘DrisStrawSeventyOne’ are more vigorous than plants of ‘35T 42’. Additionally, fruit size of ‘35T 42’ decreases more sharply later in the season than fruit size of ‘DrisStrawSeventyOne’.

‘DrisStrawSeventyOne’ differs from the proprietary male parent ‘50T403’ (unpatented) in that ‘DrisStrawSeventyOne’ has smaller, softer, and more uniformly shaped fruit compared to ‘50T403’. Moreover, ‘DrisStrawSeventyOne’ produces fewer stolons compared to ‘50T403’.

‘DrisStrawSeventyOne’ differs from the commercial variety ‘DrisStrawFortyFour’ (U.S. Plant Pat. No. 26,801) in that ‘DrisStrawSeventyOne’ has inflorescence beneath foliage, an obtuse shape of base of terminal leaflet, an outwards attitude of fruit sepals, and a partially remontant type of bearing, while ‘DrisStrawFortyFour’ has inflorescence above foliage, a rounded shape of base of terminal leaflet, an upwards attitude of fruit sepals, and a day neutral type of bearing.

‘DrisStrawSeventyOne’ differs from the commercial variety ‘DrisStrawNine’ (U.S. Plant Pat. No. 20,733) in that ‘DrisStrawSeventyOne’ has inflorescence beneath foliage, an obtuse shape of base of terminal leaflet, a free arrangement of flower petals, and a partially remontant type of bearing, whereas ‘DrisStrawNine’ has inflorescence above foliage, a rounded shape of base of terminal leaflet, an overlapping arrangement of flower petals, and a fully remontant type of bearing.

We claim:

1. A new and distinct variety of strawberry plant named ‘DrisStrawSeventyOne’ as shown and described herein.
